(19) United States
(12) Patent Application Publication
(10) Pub. No.: US 2026/0154883 A1
(43) Pub. Date: Jun. 4, 2026

(54) Inverse Geometry Tetrahedron Beam CT

(71) Applicants: Washington University, St. Louis, MO (US); TetraImaging LLC, Maryland Heights, MO (US)

(72) Inventors: Hao JIANG, Madison, WI (US); Emre TOKER, St. Louis, MO (US); Tiezhi ZHANG, Creve Coeur, MO (US); Shuang ZHOU, St. Louis, MO (US); Jonathan HAEFNER, St. Louis, MO (US); Liuxing SHEN, Maryland Heights, MO (US); Ziyu SHU, St. Louis, MO (US)

(21) Appl. No.: 19/122,139

(22) PCT Filed: Oct. 18, 2023
(86) PCT No.: PCT/US2023/077201

§ 371 Date: Apr. 17, 2025

Related U.S. Application Data
(60) Provisional application No. 63/380,121, filed on Oct. 19, 2022.
(60) Provisional application No. 63/507,151, filed on Jun. 9, 2023.

(51) Int. Cl. G06T 12/20; G16H 30/20

(52) U.S. Cl. CPC G06T 12/20; G16H 30/20

(57) ABSTRACT

This disclosure relates generally to CT x-ray imaging systems and methods, and particularly to an inverse geometry with tetrahedron beams. Specifically, a dose efficient and controllable inverse geometry tetrahedron beam CT imaging is disclosed. The disclosed CT geometry includes a linear array of x-ray sources perpendicular to a rotation axis of the CT system and rotates together with a linear detector array extended parallel to the rotation axis. The x-ray emission from each of the sources is collimated into a fan-shaped beams projected to the linear detector array. The intensity of each beam is dynamically adjusted and controlled during the rotation of the source array and the detector array. Such a configuration provides control of x-ray intensity over a selected region of interest in a target object during the rotation even if the region of interest is off from the rotation axis, with efficient dose control.

### CROSS REFERENCE

This application is based on and claims the benefit of priority to U.S. Provisional Patent Application No. 63/380,121, filed on Oct. 19, 2022, and U.S. Provisional Patent Application No. 63/507,151, filed on Jun. 9, 2023, which are herein incorporated by reference in their entireties

### GOVERNMENT SUPPORT

This invention was made with government support under grants Nos. R41DE029727 and R42EB026401 awarded by National Institute of Health. The U.S. government has certain rights in the invention.

### BACKGROUND

### Technical Field

This disclosure relates generally to computed tomography (CT) x-ray imaging systems and methods, and particularly to an inverse geometry with tetrahedron beam for CT x-ray imaging.

### Background Technologies

X-ray sources and detectors may be configured and arranged in various geometries for CT x-ray imaging. With an emergence of new types of x-ray sources and detectors, new geometries may be designed for better controlling x-ray dosage while enhancing CT image resolution and contrast in regions of interest in a target object.

### BRIEF SUMMARY

This disclosure relates generally to CT x-ray imaging systems and methods, and particularly to an inverse geometry with tetrahedron beams. Specifically, a dose efficient and controllable inverse geometry tetrahedron beam CT imaging is disclosed. The disclosed CT geometry includes a linear array of x-ray sources perpendicular to a rotation axis of the CT system and rotates together with a linear detector array extended parallel to the rotation axis. The x-ray emission from each of the sources is collimated into a fan-shaped beams projected to the linear detector array. The intensity of each beam is dynamically adjusted and controlled during the rotation of the source array and the detector array. Such a configuration provides control of x-ray intensity over a selected region of interest in a target object during the rotation even if the region of interest is off from the rotation axis, with efficient dose control.

In some example implementations, a computed tomography x-ray imaging system for imaging a target object is disclosed. The system may include a gantry frame configured to rotate around the target object with respect to a rotation axis; an x-ray source array disposed on the gantry frame, the x-ray source array comprising a plurality of x-ray sources aligned in a direction perpendicularly to the rotation axis; an elongated linear detector array disposed on the gantry frame opposing the x-ray source array with respect to the rotation axis, the elongated linear detector array comprising a plurality of x-ray detectors linearly extending along the rotation axis; a multi-slot collimator disposed on the gantry frame between the x-ray source array and the target object; and a circuitry configured to control an activation of the plurality of x-ray sources to generate a sequence of x-ray emissions which are further collimated by the multi-slot collimator into a sequence of fan beams projected through the target object into the elongated linear detector array for measurement, and the sequence of the sequence of fan beams intersecting the target object to form a tetrahedron imaging volume.

In the example implementations above, the circuitry is further configured to dynamically adjust intensities of the sequence of x-ray emissions individually.

In any one of the example implementations above, the intensities of the sequence of x-ray emissions are dynamically adjusted to accumulatively favor a projected amount of x-rays passing through a region of interest in the target object.

In any one of the example implementations above, control signals for adjusting the intensities of the sequence of x-ray emissions is based on a precomputation according to the region of interest in the target object.

In any one of the example implementations above, the intensities of the sequence of x-ray emissions are dynamically adjusted based on attenuation of the sequence of fan beams by the target object.

In any one of the example implementations above, the system further includes an elongated bowtie filter covering the plurality of x-ray sources to generate a predefined intensity profile along the rotation axis in each of the sequence of fan beams.

In any one of the example implementations above, the system further includes an elongated bowtie filter covering the plurality of x-ray sources to generate a predefined intensity profile along the rotation axis in each of the sequence of fan beams.

In any one of the example implementations above, the gantry frame together with the x-ray source array, the elongated linear detector array, and the multi-slot collimator is configured to rotate around the rotation axis to detect projections of multiple sequences of fan beams through the target object, and the circuitry is further configured to reconstruct a computed tomography x-ray image of the target object from the detected projections.

In any one of the example implementations above, the plurality of x-ray sources may be disposed to form a curved line in a plane perpendicular to the rotation axis, the curved line having a center located at the elongated linear detector array.

In any one of the example implementations above, the plurality of x-ray sources may include a linear array of x-ray sources.

In any one of the example implementations above, the multi-slot collimator comprises a plurality of slot openings each associated with one of the plurality of x-ray sources for generating one of the sequence of fan beams.

In any one of the example implementations above, the activation of the plurality of x-ray sources repeats with a repetition rate matching a frame rate of the elongated linear detector array.

In some other example implementations, a method for producing a computed tomography x-ray image of a target object is disclosed. The method may include sequentially controlling an activation of a plurality of x-ray sources to generate a sequence of x-ray emissions while rotating a gantry frame around the target object with respect to a rotation axis, the plurality of x-ray sources being disposed on the gantry frame and aligned in a direction perpendicularly to the rotation axis; collimating the sequence of x-ray emissions into a sequence of fan beams; projecting the sequence of fan beam through the target object into an elongated linear detector array, the elongated linear detector array being disposed on the gantry frame opposing the plurality of x-ray sources with respect to the rotation axis and comprising a plurality of x-ray detectors linearly extending along the rotation axis, and the sequence of fan beams intersecting the target object to form a tetrahedron imaging volume; and measuring, while rotating the gantry frame, amounts of multiple sequences of fan beams after in order to produce the computed tomography x-ray image of the target object.

### BRIEF DESCRIPTION OF THE DRAWINGS

FIG. 1 illustrates an example CT system.

FIG. 2 illustrates a general inverse geometry CT with tetrahedron beams.

FIG. 3 illustrates a perspective view of an inverse geometry tetrahedron beam CT with a linear array of x-ray sources, a linear detector array, a multi-slot collimator and a bowtie filter.

FIG. 4 shows a top view of the inverse geometry tetrahedron beam CT of FIG. 3.

FIG. 5 shows a side view of the inverse geometry tetrahedron beam CT of FIG. 1.

FIG. 6 illustrates an example for sequentially scanning x-ray sources in a source array.

FIG. 7A and FIG. 7B illustrate off-axis region of interest selection by dynamically controlling intensity profile of x-ray sources.

FIG. 8 illustrates example of CT images reconstructed with uniform x-ray fluence and with intensity modulation based on a selected region of interest.

FIG. 9 illustrates an inverse geometry tetrahedron beam CT configuration with stacked x-ray source arrays.

FIG. 10 illustrates an inverse geometry tetrahedron beam CT configuration with an axial offset between x-ray source array and detector array.

FIG. 11 illustrates an example multipixel thermionic emission x-ray (MPTEX) source with a control circuitry.

FIG. 12 illustrates an example relationship between a driving voltage and emission current of electrons for a thermionic filament cathode under an example negative bias.

### DETAILED DESCRIPTION

Various aspects for CT imaging will now be described in detail hereinafter with reference to the accompanied drawings, which form a part of the present disclosure, and which show, by way of illustration, various example implementations and embodiments. The systems, devices, and methods for configuring x-ray source and detector arrays in an inverse geometry to achieve tetrahedron volumetric field of view with dynamically adjustable x-ray intensity profiles, as disclosed herein, may, however, be embodied in a variety of different forms and, therefore, the disclosure herein is intended to be construed as not being limited to the embodiments set forth below. Further, the disclosure may be embodied as methods, components, and/or platforms in addition to the disclosed devices and systems. Accordingly, embodiments of the disclosure may, for example, take the form of hardware, software, firmware or any combination thereof.

In general, terminology may be understood at least in part from usage in its context. For example, terms, such as “and”, “or”, or “and/or,” as used herein may include a variety of meanings that may depend at least in part upon the context in which such terms are used. Typically, the term “or”, if used to associate a list, such as A, B or C, is intended to mean A, B, and C, here used in the inclusive sense, as well as A, B or C, here used in the exclusive sense. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for the existence of additional factors not necessarily expressly described, again, depending at least in part on context.

Many other modifications of the implementations above may be made to adapt a particular situation or material to the teachings without departing from the scope of the current disclosure. Therefore, it is intended that the present methods and systems not be limited to the particular embodiments explicitly disclosed. The disclosed methods and systems include all embodiments falling within the scope of the appended claims.

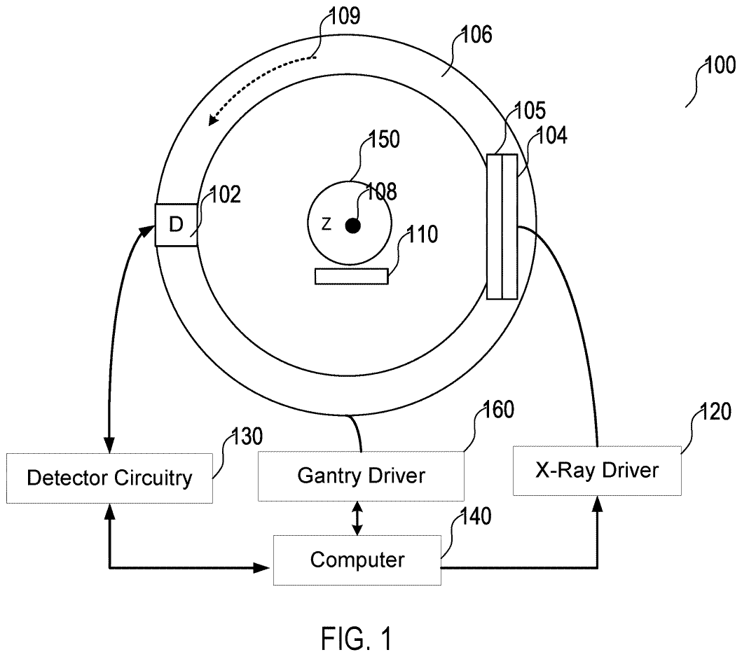

By way of introduction, Computed Tomography (CT) is a critical tool for diagnostics in medical imaging. An example CT system 100 is shown in FIG. 1. The example CT system 100 may include a gantry frame 106 configured to rotate around axis 108, also labeled as “z”, in an example direction shown by arrow 109. The example CT system 100 further includes a couching platform 110 for supporting a target object 150 during CT image acquisitions. The example CT system 100 further includes an x-ray source assembly 104 disposed on or in the gantry frame 106 and an x-ray detector assembly 102 disposed on or in the gantry frame 106 on an opposing side of the x-ran source assembly with respect to the rotation axis 108, such that x-ray beams emitted from the x-ray source assembly 104 are projected through the target object 150 to the x-ray detector assembly 102. In the configuration of FIG. 1, the gantry frame along with the x-ray source assembly 104 and the x-ray detector assembly 102 disposed therein/thereon may be configured to rotate around the fixed couching platform 110 and the axis 108.

In some example implementations, the gantry frame 106 along with the x-ray source assembly 104 and the x-ray detector assembly 102 disposed therein/thereon may be further configured to translate along the z direction for projection coverage of the target object along the z direction.

In some example implementations, the CT system 100 may further include one or more x-ray beam shapers 105 disposed in front of the x-ray source assembly 104 to shape the x-ray emission from the x-ray source assembly 10. The shaping of the x-ray emission may include but is not limited to collimating, fanning, directioning, intensity profiling, and the like. Examples such as bowtie filters and slot collimators are described in further detail below.

The example CT system 100 further includes an x-ray driver circuitry 120 for activating and controlling emission of the x-ray beam(s) in the x-ray source assembly 104. The example CT system 100 further includes a detector circuitry 130 for controlling the detection of the projected x-ray beams and for collecting detected amount of x-ray. The example CT system 100 further includes a gantry driver 160 for controlling the rotation and/or translation of the gantry frame 106. The detector circuitry 130, the x-ray driver circuitry 120, and the gantry driver 160 may be in communication with one or more central or distributed computers 140 which provide control of the operation of the x-ray source assembly 104, the x-ray detector assembly 102, and the gantry, as well as computation and reconstruction needs for the CT system 100.

During operation of the example CT system 100, the one or more computers 140 may be configured to control the x-ray source assembly 104 to emit x-ray beam(s) which may be shaped by the-ray beam shaper 105 and projected through the target object 150 to the x-ray detector assembly 102 for measurement. Multiple projections may be detected while the gantry is rotated around and/or translated along the rotation axis 108. The multiple projections accumulatively cover a volumetric region of the target object and are then reconstructed by the one or more computers to form CT images.

In the example CT system 100 above, the design/control of the x-ray source assembly 104 and the x-ray detector assembly 102 may greatly affect the x-ray flux required, the speed of image acquisition, and the quality of the reconstructed images.

For example, in a traditional CT system, a point x-ray source may be employed in the x-ray source assembly 104. The x-ray beam shaper 105 may be configured to shape the emission from the point x-ray source in a single direction beam. As such, each project from the x-ray source assembly 104 to the detector 102 only covers a line direction through the target object. In order to obtain projections to cover the imaging volume in a reasonable amount of time, the gantry frame 106 may be rotated of a relatively high speed and translated along the rotation axis while firing or activating the x-ray source assembly 104, with each activation generating one projection. Such a system may be reference as a helical CT scanner. A helical CT scanner can produce high quality images but may be too cumbersome for many applications that are more than just for diagnostic purposes, such as CT systems that also incorporate treatments, e.g., point-of-care imaging systems and image guided intervention systems.

In some other example implementations of the point x-ray source, the x-ray beam shaper 105 may be configured to produce a planar fan beam, which projects through the target object to the x-ray detector assembly 102. The x-ray detector assembly 102 would then correspondingly include a detector array, e.g., a linear detector array having a plurality of detectors linearly aligned in single or multiple rows in the plane of the fan beam for pixelated detection. As such, each projection of a fan beam would pass through a plane (rather than a line) in the target object, allowing for simultaneous detection of multiple project lines in the fan beam, thereby significantly increasing the speed of the data acquisition or requiring slower rotation speed for the gantry frame 106.

A collimator may be used as the x-ray beam shaper for shaping an emission from a point x-ray source into a fan beam. For example, the collimator may be made based on a slot on a block of radiopaque materials such as brass.

The fan beam may be further processed by a bowtie filter to generate a predefined intensity profile within the fan plane of the fan beam. As described in further detail below, such intensity profile may facilitate concentrating x-ray flux to regions of the target object that is of higher interest. Examples of using bowtie filter are provided in further detail below.

In some other example implementations of the point x-ray source, the x-ray beam shaper 105 may be configured to produce a cone beam, which projects through the target object 150 to the x-ray detector assembly 102. The x-ray detector assembly 106 would then correspondingly include a detector array, e.g., a 2-D detector array or flat panel having a plurality of detectors arranged in two-dimensions to receive the projected cone beam. As such, each projection of a cone beam would cover a conical volume (rather than a plane or a line above) in the target object, allowing for simultaneous detection of multiple project lines in the cone beam, thereby significantly increasing the speed of the data acquisition or requiring slower rotation speed for the gantry frame. The term “detector array” or “array detector” or “array detectors” are used interchangeably to refer to the assembly or collection contain the multiple x-ray detectors.

A CT system based on point source and cone beams may have a compact geometry and can scan a large volume with a single system rotation by requiring a 2-D detector. However, cone-beam CT systems may not be capable of producing high image quality due to the excessive x-ray scattering and sub-optimal performance of typical flat panel 2-D x-ray detectors.

In some other example implementations, the CT system above may use an inverted geometry. The term “inverted” may be used refer to the reversion of projection between the x-ray source and the x-ray detector, particularly in the context of the fan beam or cone beam geometry above. For example, in the context of the fan beam geometry, rather than having a single x-ray source to generate a fan beam and a linear array of detector for detecting different portions of the fan beam, an array of x-ray line beams may be generated by the x-ray source assembly 104 and are projected into a single pixel detector in the x-ray detector assembly 102. Such an inverted geometry nevertheless covers identical projected plane in the target object except that a plurality of x-ray sources in the source array would fire in a time sequence, that each firing provides one-line projection, and that the measurement by the single detector of each line projection is time-resolved from other projection lines.

Such inverted geometry allows for using of high-sensitivity single pixel x-ray detectors (e.g., photon counters) and would be beneficial when compact linear array of x-ray sources are readily available.

In the example inverse geometry implementations above, the x-ray source may be expanded to 2-dimensional. As such, a projection volume from the detector array to the single pixel detector may be covered by sequentially firing the individual x-ray sources in the array and time-resolve their detection in the single-pixel detector after being projected.

In some other example implementations for volumetric CT, both the x-ray source assembly 104 and the x-ray detector assembly 102 may contain an array of individual components. For example, the x-ray source assembly 104 may include a multiple individually activatable and controllable x-ray sources, and the x-ray detector assembly 1024 may include multiple pixelized x-ray detectors. Specifically, both arrays may be linearly arranged: the x-ray detector assembly may include an extended line of x-ray detectors whereas the x-ray source assembly may include an extended line of x-ray sources.

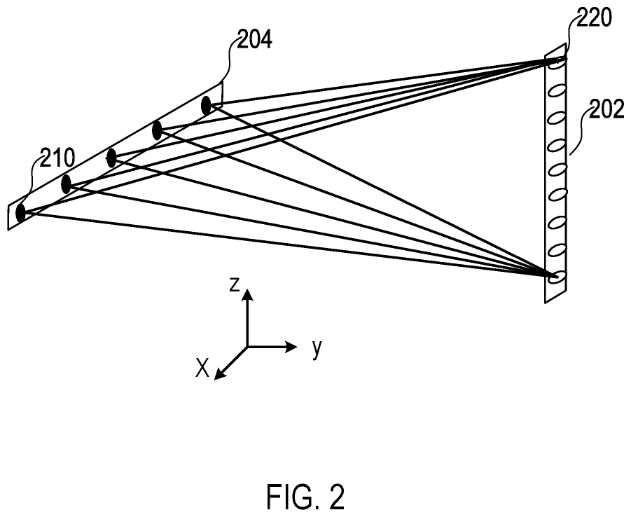

In some example implementations, the two linear assemblies may be arranged or disposed on the gantry frame such that they are perpendicular to each other, as shown in FIG. 2. Specifically, FIG. 2 shows a linear detector array 202 and a linear source array 204. X-ray emission for each of the sources, such as source 210, may be shaped into a planar fan beam projected through the target object (not shown in FIG. 2) to cover the detector array 202. Similar to other systems based on source array, the x-ray sources of FIG. 2 are individually activatable and controllable. They may be activated sequentially such that their fan beams are sequentially projected and detected by the detector array 202 and are thus time-resolved. The N detectors (N being an integer) in the detector array 202 further resolve N lines in each of the fan beams spatially. As such, each of the fan beams covers a line in the target sample and a sequence of activation of all the sources in the x-ray source array 204 accumulatively covers a volumetric region of the target object. The volumetric region of the target object is defined by the intersection of all the fan beams and the target object. As can be seen from FIG. 2, such a volumetric region is tetrahedron in nature in the z and x direction, and is determined by the object boundary in the y direction of FIG. 2. This geometry is thus referred to as tetrahedron geometry, and a CT system employing such a geometry may be referred to as a tetrahedron beam CT system.

The example geometry of linear detector and source array is also an inverse geometry in that all the x-ray sources in the source array 204 projects to each of the detector unit in the detector array 202, such as detector 220. As such, a CT system employing the geometry illustrated in FIG. 2 may be alternatively referred to as an inverse geometry tetrahedron CT system. Tetrahedron Beam CT may advantageously produce similar high-quality images as helical CT above in a compact geometry similar to cone-beam CT above. The term detector unit, detector pixel, individual detector, and the like may be interchangeably used.

In one particular example implementation of FIG. 2, the source array 204 may be disposed in the gantry frame 106 of FIG. 1 to extend parallel to the rotation axis 108 whereas the detector array 202 is disposed in the gantry frame 106 to extend perpendicular to the rotation axis 108. The gantry frame 106 may slowly rotate such that there is negligible motion blur during each sequence of x-ray beam activations or scan in the source array. In one gantry frame rotation, multiple sequences may be scanned in the source array, correspondingly forming multiple tetrahedron volume projections. The time and spatially resolved measurements of these projections may then be reconstructed by the one or more computers 140 into CT images.

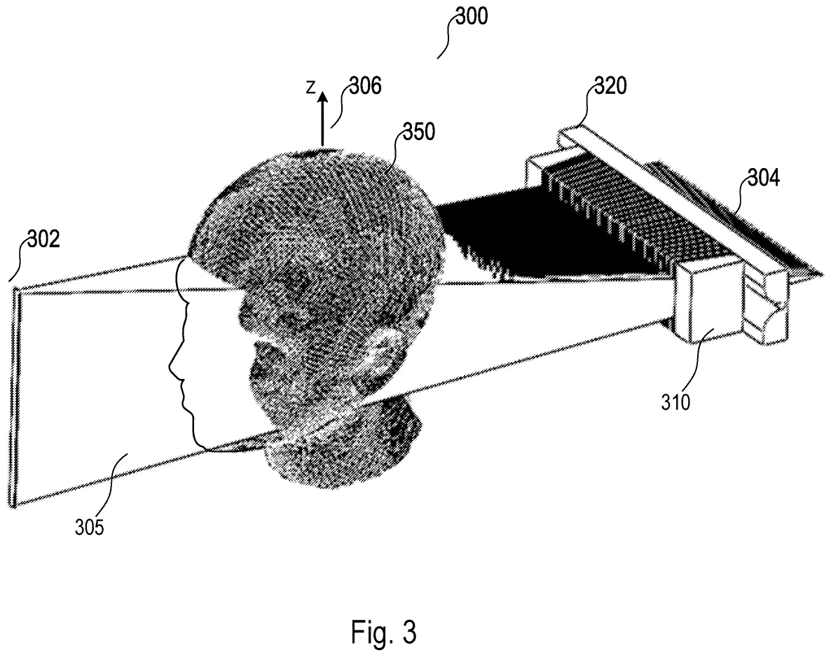

In another particular example implementation of FIG. 2, the source array 204 may be disposed in the gantry frame 106 of FIG. 1 to extend perpendicular to the rotation axis 108 whereas the detector array 202 is disposed in the gantry frame 106 to extend parallel to the rotation axis 108, as illustrated in FIG. 3. In FIG. 3, the inverse geometry tetrahedron beam CT system 300 includes a linear array x-ray sources 304, a linear array detector 302, a bowtie filter 320 and a multi-slot collimator 310. The bowtie filter 320 and the multi-slot collimator 310 function together as the x-ray beam shaper 105 of FIG. 1. The system 300 rotates about a central axis 306. The linear source array 304 is aligned perpendicular to the rotation axis 306 and the linear array detectors 302 are aligned parallel to the rotation axis 306 and perpendicular to a rotation plane.

The x-ray sources in the linear source array 304 generate diverging x-ray emissions at the focal spot positions. Emission from each of the x-ray sources is collimated to a fan-shaped x-ray beam by the multi-slot collimator 310.

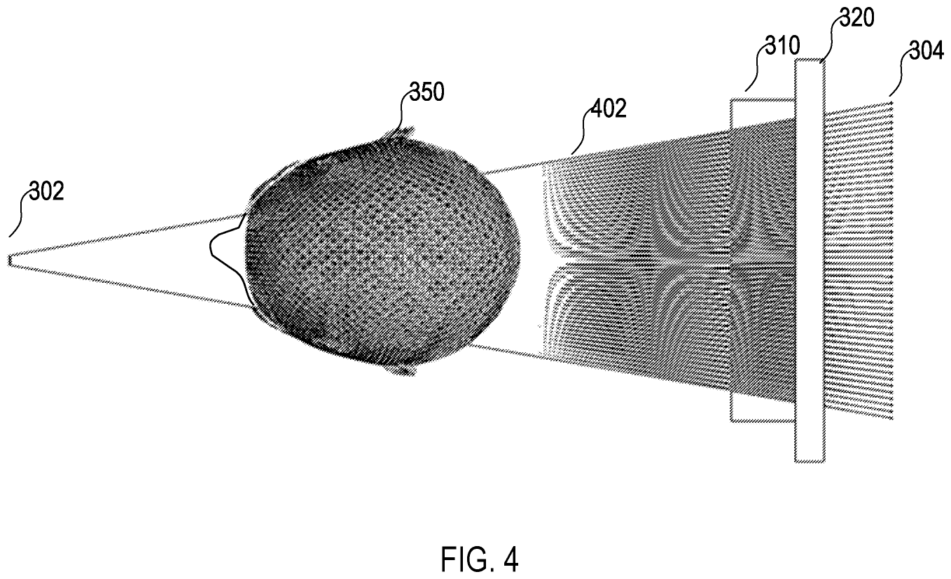

FIG. 4 shows a top view of the example embodiment of FIG. 3. As shown in FIG. 4, again, the x-ray beams generated by the linear x-ray sources are collimated into a stack of fan-shaped beams 402 by the multi-slot collimator 310, passing or projected through the imaging or target object 350, and then received by the linear array detector 302.

The multi-slot collimator 310 may include slanted slots that converge the x-ray beams to the detector 302, as shown in FIG. 4. The multi-slot collimator can be made by machining slant slots on a block of radiopaque materials such as brass or can be made by machining slant slots a block of radiotransparent materials, which are inserted with radiopaque plates.

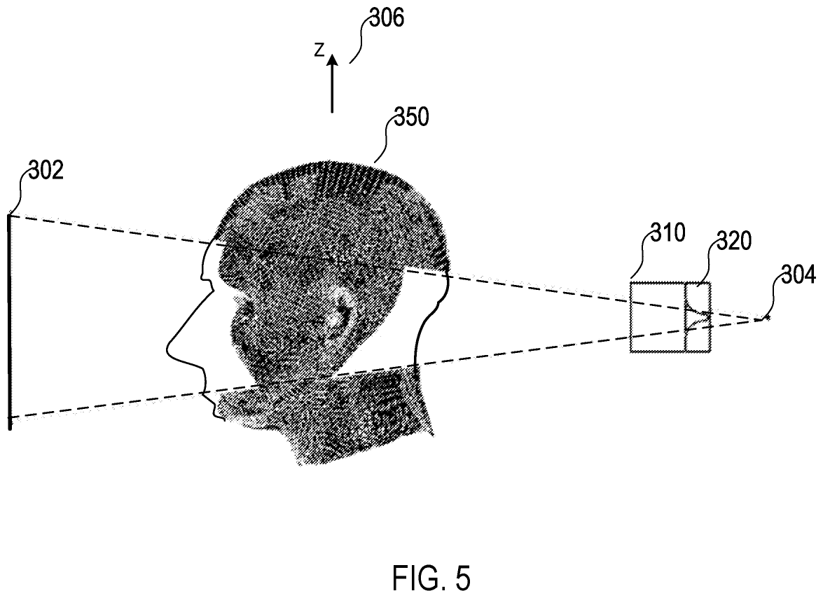

FIG. 5 shows a lateral or side view of the example embodiment of FIG. 3 to illustrates the function of the bowtie filter 320 in further clarity. The x-ray emissions from the sources pass through the bowtie filter 320 before reaching the multi-slot collimator 310. The bowtie filter 320 allows more x-ray flux to pass in the center of the emission, as shown by the example transmission profile indicated in FIG. 5 within 320. As such, each fan beam may carry such intensity profile in its fan plane parallel to the rotation axis 306. As such, the x-ray flux may concentrate more in the portion that project through a region of interest in the target object 350 in the axial direction.

The bowtie filter 320 may be constructed from a material that is semi-transparent to x-ray, i.e. aluminum or Teflon. The bowtie filter may be constructed in a shape that is thinner in the middle and thicker at the side, resembling a bowtie. The bowtie filter may be removable disposed in or on the gantry frame in front of the x-ray source(s) and thus may be replaceable. Bowtie filters may have different shapes that allow different lengths/ranges in z (axial direction in FIG. 5) to receive higher x-ray flux. Different bowtie filters or no bowtie filter may be chosen depending on different needs.

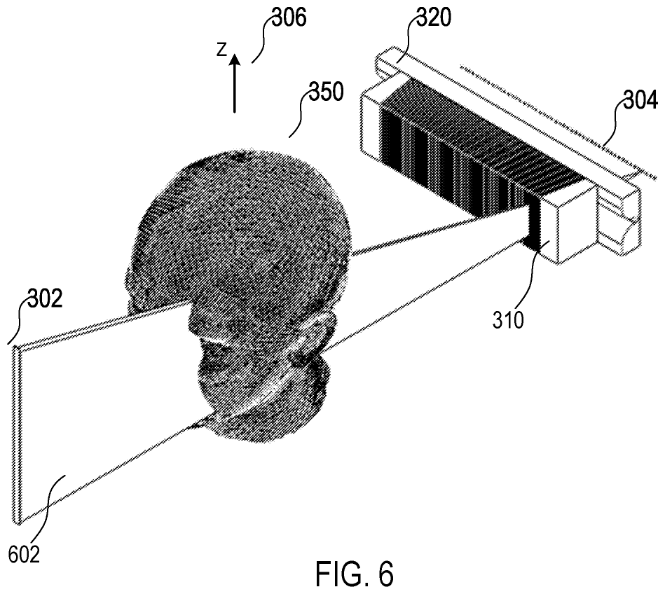

As further shown in FIG. 6, a fan beam 602 from one of the x-ray sources in the x-ray source array 304 may be activated while all other x-ray sources are turned off. The x-ray sources may be turned on or activated sequentially to generate a sequence of fan beams, as described above. The activation or scan of the sequence of the fan beams repeats as the gantry rotate slowly the rotation axis 306. The linear detector array 302 acquires a frame of projection data by each x-ray fan beam.

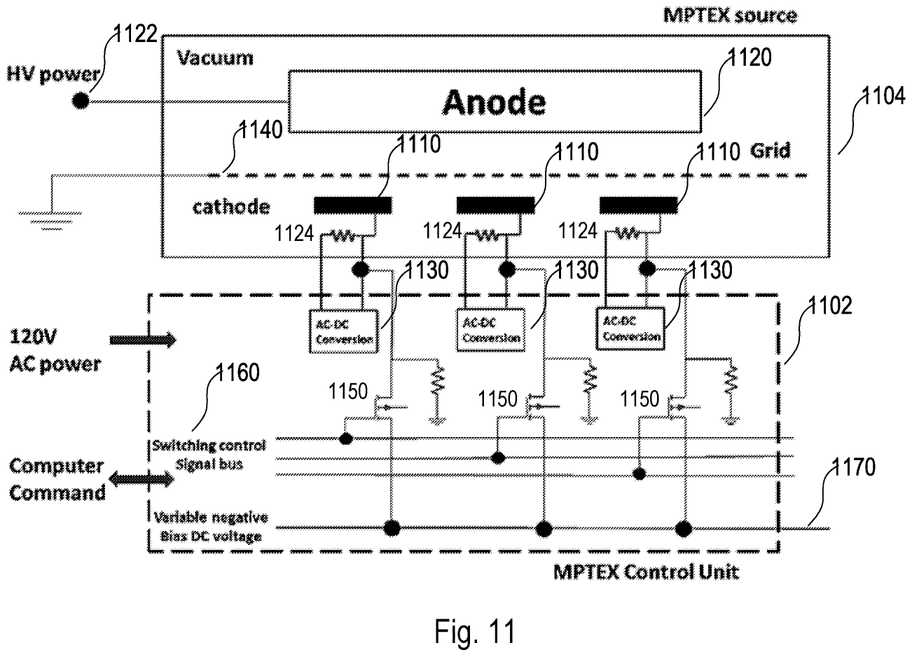

In the example inverse geometry tetrahedron implementation of FIG. 3, the intensity of the fan beams 305 may be individually controlled during the activation and scan of each x-ray source of the x-ray source array 304. An example of x-ray source array 304 and the control/adjustment of intensity of emission of each individual source is illustrated further below in relation to FIG. 11.

In some example implementations, the intensity of each of the fan beams 305 of FIG. 1 may be programmed, with higher intensity in the center beams and lower intensity off center. Such programmed intensity profile may be configured to emulate a bowtie filter described above and may be repeated (or re-programed) between scans of the x-ray sources during the rotation of the gantry frame.

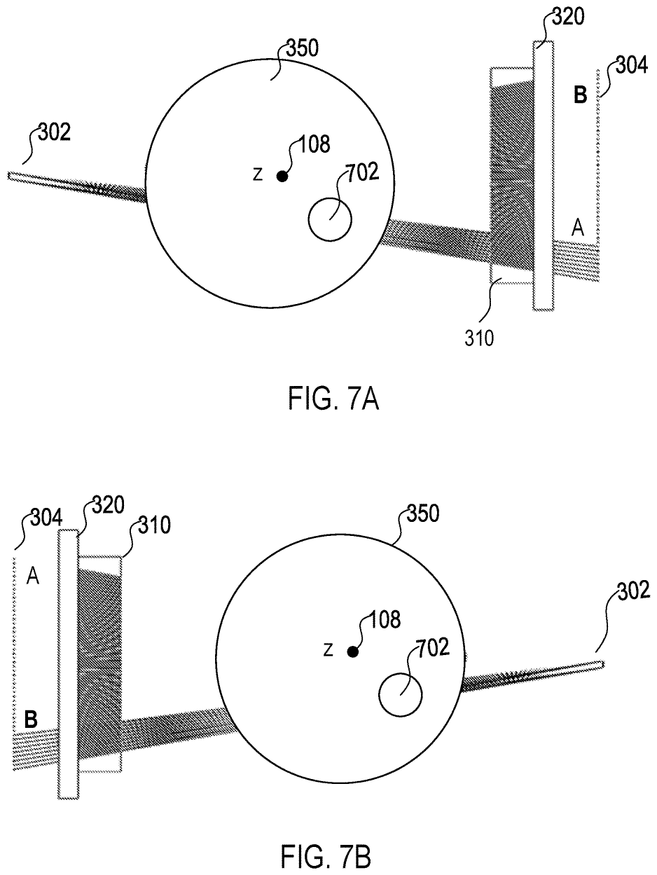

In comparison to a traditional fixed physical bowtie filter, the x-ray sources may further be dynamically reprogrammed from sequence to sequence of beams and the intensity profile between sequences may be dynamically varied. An advantage of such capability is to keep the intensity profile of the projections of the beams close to a region of interest (ROI) in the target object during the rotation of the gantry frame even if the ROI is off the rotation axis, as illustrated in FIG. 7. Specifically, as shown in FIG. 7, the ROI 702 may be off the rotation axis 108 of the gantry frame. When the gantry frame along with the x-ray sources, the collimator, and the bowtie filter, and the detector array (304, 320, 310, and 302) rotates about the rotation axis 108, the activated beams from each specific x-ray source in the source array 304 passes through different slices in the target object 350 during the rotation. If the intensity profile of the beams is repeated from beam sequence to beam sequence during the rotation, then because the target object does not rotate, the off-axis ROI 702 would not receive a same intensity. In order to keep the ROI 702 illuminated with higher intensity during the rotation of the gantry frame, the profiles within each beam sequence may be dynamically adjusted (or reprogramed). For example, as shown in FIG. 7A, at a first angular position of the rotation axis 108, x-ray beams from portion A of the x-ray sources with projects through the ROI 702 may be adjusted to have higher intensity. As the gantry frame rotates to a second angular position of the rotation axis 108, x-ray sources at position B instead would be adjusted to have higher intensity, as shown in FIG. 7B. As such, the intensity profile may be dynamically adjusted or reprogramed during the rotation of the gantry frame so as to maintain higher x-ray intensity at or near the ROI 702. Without such dynamical adjustability, such as in fixed bowtie filter, the ROI would have to be placed at the rotation axis in order for it to always receive the desired higher intensity during the rotation of the gantry frame.

In some other example implementations, the intensity of the fan beams at each sequence can be dynamically programmed during the scan of the beams in the x-ray source array, based on the maximum reading of detector 102 or 302, producing a variable intensity profile similar to a virtual bowtie filter for each scan sequence of x-ray sources in the source array. Thus, the dynamical control of the intensity profile between the fan beams effectively create a virtual bowtie filter whose intensity profile can change as programed as the gantry frame rotates around the target object. For example, the beam intensity may be modulated or adjusted according to the thickness of the slice of the target that the beam is projecting through at a particular time of the rotation of the gantry.

In some example implementations, the dynamically adjusted intensity profile may be precomputed and programed for an entire rotation of the gantry frame including multiple scan sequences of the x-ray sources. Such precomputation may be based on, for example, the location of the ROI in the target object and the speed of the rotation and the gantry frame. Such precomputation may be converted into control signals for driving the x-ray driver circuitry 120 of FIG. 1 for the sequential activation of the x-ray resources in the x-ray resource array 304 of FIG. 7.

The combination of the dynamical intensity profile adjustment/programing across projection planes parallel to the rotation axis, as described above, and the additional intensity profiling within each fan beam in the direction along the rotational axis by the bowtie filter 320 of FIGS. 3-7 (if included) advantageously provides ROI control in all three spatial dimensions in the target object.

In the other inverse geometry tetrahedron beam implementation where the detector array extends in the rotational plane and the x-ray source array extends along the rotation axis, the beam's intensity can be similarly controlled in a dynamical manner. However, such intensity profile control would be along the rotational axis. When coupled with a similar but fixed-profile bowtie filter to generate an intensity profile of each beam, only an off-axis cylindrical region with higher intensity can be achieved. As such, the geometry of FIGS. 3-7 provides more selective off-axis ROI in the target object.

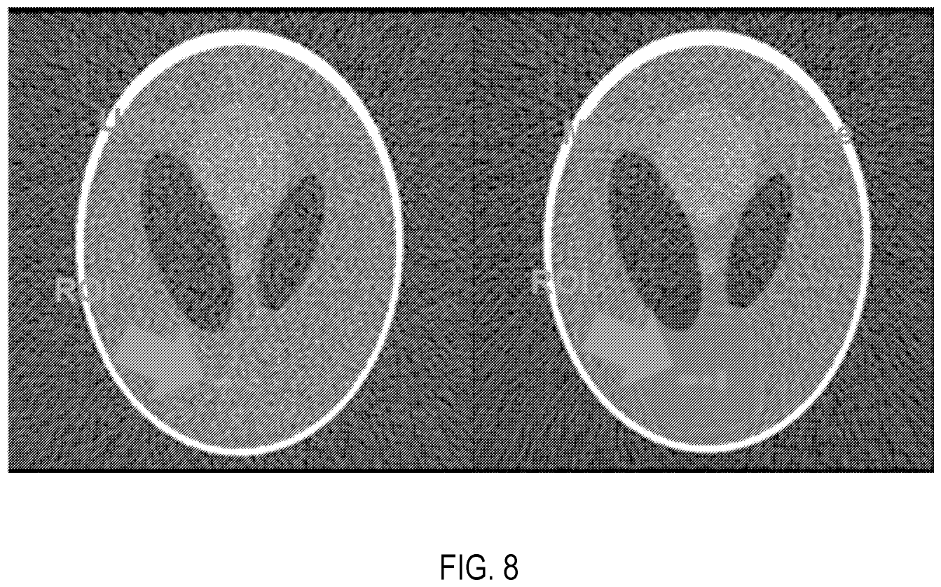

The right panel of FIG. 8 shows simulated results of images reconstructed by dynamically modulated fluence based on ROI locations produced by the inverse geometry tetrahedron implementation of FIGS. 3-7, as compared to images in the left panel of FIG. 8 by leaving the x-ray fluence profile uniform. Due to the concentrated x-ray fluence, the ROI in the image region as indicated by the arrows in FIG. 8 has better visibility using the geometry of FIGS. 3-7 than using uniform x-ray fluence.

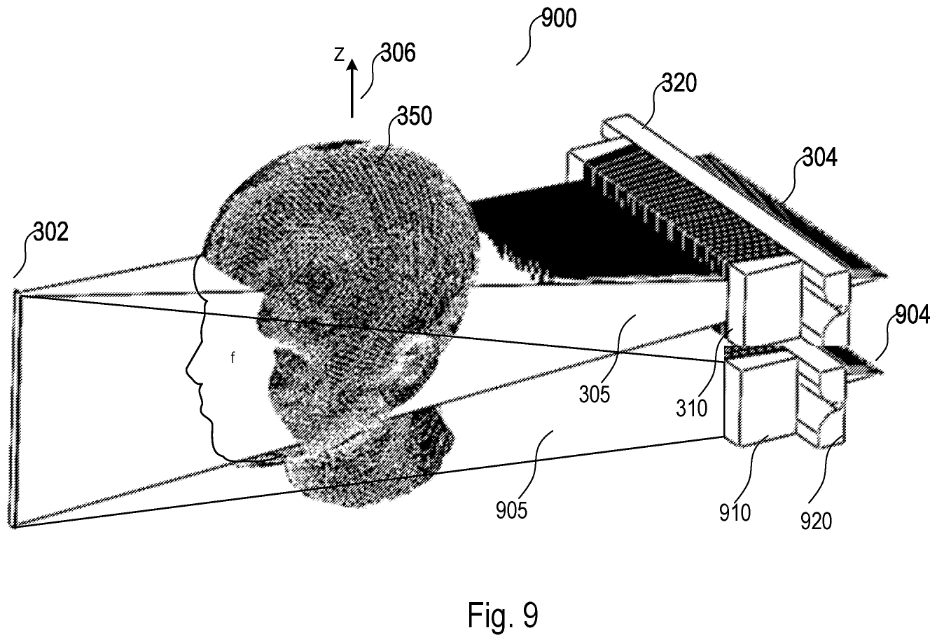

FIG. 9 illustrates another inverse geometry tetrahedron beam CT system 900. In comparison to the implementations of FIGS. 3-7, the system 900 includes multiple, e.g., two x-ray source arrays 304 and 904. Correspondingly, the system 900 also includes, for example, two bowtie filters 320 and 920, as well as two slanted multi-slot collimators 310 and 910. The two x-ray source arrays thus generate two sets of x-ray emissions that are filtered by bowtie filters 320 and 920, and then collimated by the multi-slot collimators 310 and 910 to generate two sequences of x-ray fan beams 305 and 905. The two linear source arrays help increase the imaging field of view in axial direction and may additionally or alternatively provide stereoscopic view of the anatomy in the center of the target object.

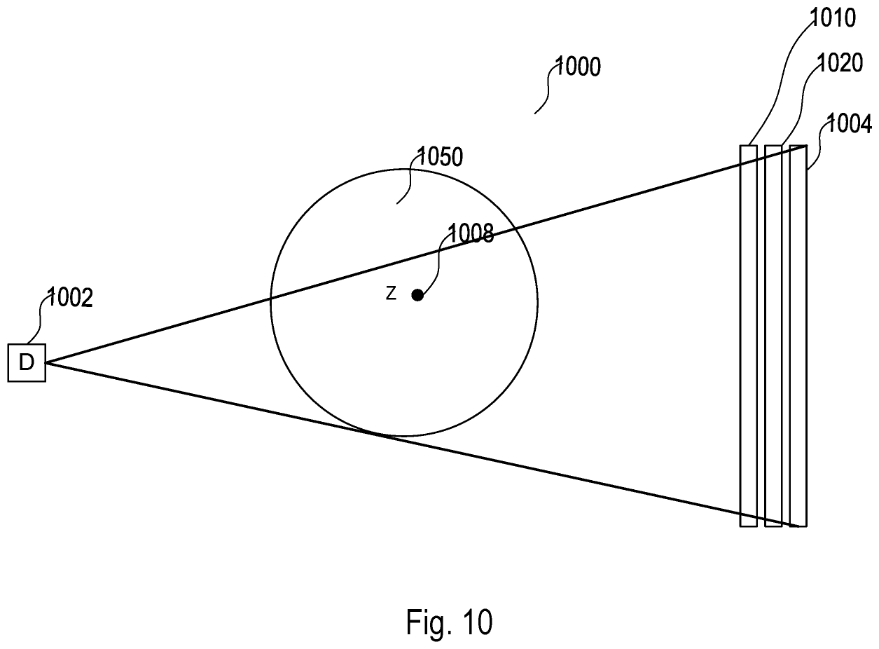

FIG. 10 further illustrates another inverse geometry tetrahedron beam CT system 1000 that follows the geometrical and operational principles of the implementation of FIGS. 3-7, but with the linear array x-ray source 1004 and linear array detector 1002 in an offset position relative to the rotation axis 1008. The offset of the source and detector position allows for reconstruction of a larger field of view with the source array of similar sizes. A large reconstruction field of view can be achieved by offset the source array only, the detector array only, or both.

In any one of the implementations above, the linear x-ray source array 104, 304, 904, and 1004 of FIGS. 1-7 and 9-10 can also be curved, for example, in a partial arc with a center located at the detector position. The detector array 102, 302, and 1002 can also be curved, for example, in a partial arc with a center located at the source array position.

The linear array detector 104 may be implemented as a multi-row x-ray detector array. In some example implementations, detector pixel size can be very small, e.g., 0.1-1 mm, but an x-ray fan beam cannot be so thin. A multi-row detector that is not too wide may be used to detect the fan beam. For example, detector 104 may be a 6 mm wide, 15 cm long detector that contains 60×1500 detector pixels.

Anti-scatter grid may be used to further reject scattered x-ray photons. Anti-scatter grid may be 1-D or 2-D, and positioned in front of the detector 104, to reject scattered photons from the subject 150.

The linear x-ray source arrays 102, 304, 904, and 1004 of FIGS. 1-7 and 9-10 above may be implemented in various manners for facilitating dynamic control of emission intensity from each individual x-ray source in the array in a sufficiently fast time scale during the rotation of the gantry frame and for fast on/off control of the x-ray emission for the sequential activation and scan of the sources as described above. For example, such an x-ray source array may be implemented as a multipixel thermionic emission x-ray (MPTEX) source comprising a plurality of thermionic electron sources, as shown in FIG. 11. For example, such an MPTEX may include 20-100 (e.g., 50) thermionic electron sources 1110. The x-ray flux generation from each thermionic electron source 1110 may be controlled by varying heating powers for its filament 1124, and by varying the bias voltage 1170 between the electron source 1110 and the gate 1140. The x-ray emission can be turning on and off the current in the filament. Controlling the emission flux or intensity may be achieved by varying the heating/driving current or power 1130 to the heating filaments, and/or by pulse width modulation (by varying duty cycle) in the driving current for the electron sources. When one source is controlled to emit x-ray, other sources in the array is controlled to turn off by switch 1150.

For further details, FIG. 11 shows an example MPTEX vacuum tube (1104) and control unit (dashed line box 1102). The MPTEX vacuum tube 1104 may include, for example 50 cathodes 1110 (only three of them are illustrated in FIG. 11 as representatives) that, for experimental purposes, correspond to 50 focal electron spots on an elongated fixed anode 1120 for creating 50 point-sources of x-rays. The anode voltage 1122 is provided, for example, by a commercial 6 KW, 120 kV high voltage power supply (e.g., Model STR120, Spellman High Voltage Electronics). Each heating filament 1124 acts as a cathode is powered by an isolated and thus individually controllable DC power 1130. With a grounded mesh grid 1140 in the MPTEX tube 1104, beam on and off can be controlled by switching in and out a negative bias voltage between the cathodic heating filaments 1124 and the ground grid. For example, for turning the electron emission on, the negative bias voltage from line 1170 is connected via controlling the switches 1150 using control signal 1160 to pass the bias voltage 1170 to the heating filament. When the negative bias is applied (on state for a source), the electron flus is controlled by the DC voltage from the power supply 1130. For turning the current in the filament off, the switches 1150 may be gated by the control signal 1160 to disconnect the negative bias voltage 1170 to the heating filament. As such, at least two different controls over the emission of the x-rays by MPTEX can be achieved. For example, the electron emission on/off can be controlled by controlling the bias voltage via the switches 1150, and the electron emission flux can be controlled by the driving voltage from the power supplies 1130 when the bias is applied.

Specifically, in the example of FIG. 11, the power of each cathode may be provided by a variable power supply. The output parameters of the variable power supplies may be controlled by a microcontroller unit (MCU). By programming the MCU, the heating power or current of each channel can be adjusted dynamically during the inverse-geometry CT scan when the channel (cathode) is biased to turn on.

Burnout of filament cathodes such as 1124 of FIG. 11 may be a likely failure mode of the MPTEX sources. Thus, it is important to minimize the working temperatures of the cathodes during CT scans. The heating powers or may be controlled by the MCU, which communicates with the controllers of the variable power supplies. The voltages of each variable power supply can be adjusted in milliseconds, sufficient for dynamic intensity control during the rotation of the gantry frame. The bias control for switching on/off of the cathode current, on the other hand, can be as short as in micro seconds, thus sufficiently fast for scanning from cathode to cathode. It may even sufficiently fast for implementing a pulse-width modulation to achieve additional or alternative flux/intensity control by adjusting a pulse width duty cycle during the time slot for a particular source of the array.

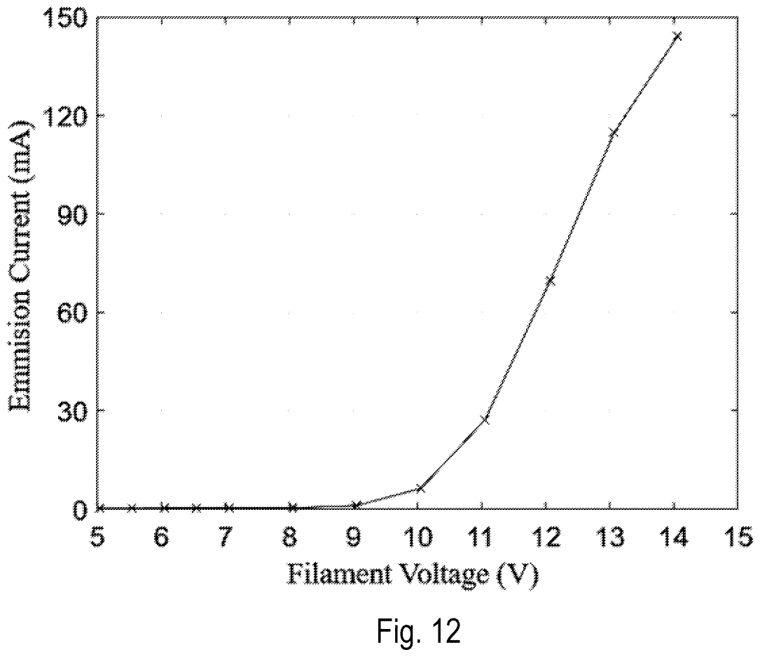

With respect to the dynamic intensity control vial adjusting the driving power to the filaments, FIG. 12 shows an example measured relationship between emission current and filament voltage for a typical filament. With a fixed −200V bias voltage between filament and grounded mesh grid, emission current starts to ramp up at 9V filament voltage with an emission current of 1.05 mA. The corresponding filament current (not shown) is 2.11 A and the power of the filament is ˜18 W. The emission current rapidly increases as filament voltage further increases, and gradually reaches 144 mA at 14V. The MPTEX source operates between 5-30 mA during inverse-geometry CT scans. Therefore, the filament voltage varies between 9 to 11V during inverse-geometry CT scans, as programmed by the MCU.

In the reverse geometry tetrahedron beam CT implementations above, the rotation of the gantry frame may be at several second to several tens of second per rotation. The scan of the sources within the source array per source may be at microsecond to millisecond time scale. There may be tens to hundreds of sources in the array, thereby taking tens of microseconds to hundreds of milliseconds to complete one sequence. As such, many source scans or sequences may be had during one gantry rotation. For example, between tens of to thousands of scans or sequences may be had during one rotation.

Finally, the advantages of the reverse geometry tetrahedron beam CT implementations above, particular the ones illustrated in FIGS. 3-7 and 9-10, include, for example, a significant reduction of the radiation dose to the peripheral tissues and organs outside the region of interest due to the dynamic intensity profile control of the individual sources. The implementations above allows for off axis ROI selection, which is not achievable using traditional bowtie filters. The intensity of the beams can also be adjusted according to the thickness of the target object along the slice associated with the particular beam at a particular time. The implementations above enables more efficient utilization of the x-range doses.

It is to be understood that the various implementations above are not limited in its application to the details of construction and the arrangement of components set forth above and in the accompanying drawings. The disclosure is intended to cover other embodiments that may be practiced or carried out in various ways following the underlying principles disclosed herein.

It should also be noted that a plurality of hardware and software-based devices, as well as a plurality of different structural components may be used to implement the various embodiments of the disclosure. In addition, it should be understood that embodiments of this disclosure may include hardware, software, and electronic components or modules that, for purposes of discussion, may be illustrated and described as if the majority of the components are implemented solely in hardware. However, one of ordinary skill in the art, and based on a reading of this disclosure, would recognize that, in at least one embodiment, the electronic based aspects of the invention may be implemented in software (e.g., stored on non-transitory computer-readable medium) executable by one or more processors. As such, it should be noted that a plurality of hardware and software-based devices, as well as a plurality of different structural components may be utilized to implement the invention. Furthermore, and as described in subsequent paragraphs, the specific mechanical configurations illustrated in the drawings are intended to exemplify embodiments of the invention and that other alternative mechanical configurations are possible. For example, “controllers” described in the specification can include standard processing components, such as one or more processors, one or more computer-readable medium modules, one or more input/output interfaces, and various connections (e.g., a system bus) connecting the components. These controllers may be implemented as dedicated processing circuitry or in general-purpose processors, in combination of various software and/or firmware, and in combination of other wired or wireless communication interfaces.

We claim:

1. A computed tomography x-ray imaging system for imaging a target object comprising:
a gantry frame configured to rotate around the target object with respect to a rotation axis;
an x-ray source array disposed on the gantry frame, the x-ray source array comprising a plurality of x-ray sources aligned in a direction perpendicularly to the rotation axis;
an elongated linear detector array disposed on the gantry frame opposing the x-ray source array with respect to the rotation axis, the elongated linear detector array comprising a plurality of x-ray detectors linearly extending along the rotation axis;
a multi-slot collimator disposed on the gantry frame between the x-ray source array and the target object; and
a circuitry configured to control an activation of the plurality of x-ray sources to generate a sequence of x-ray emissions which are further collimated by the multi-slot collimator into a sequence of fan beams projected through the target object into the elongated linear detector array for measurement, and the sequence of the sequence of fan beams intersecting the target object to form a tetrahedron imaging volume.

2. The computed tomography x-ray imaging system of claim 1, wherein the circuitry is further configured to dynamically adjust intensities of the sequence of x-ray emissions individually.

3. The computed tomography x-ray imaging system of claim 2, wherein the intensities of the sequence of x-ray emissions are dynamically adjusted to accumulatively favor a projected amount of x-rays passing through a region of interest in the target object.

4. The computed tomography x-ray imaging system of claim 3, wherein control signals for adjusting the intensities of the sequence of x-ray emissions is based on a precomputation according to the region of interest in the target object.

5. The computed tomography x-ray imaging system of claim 2, wherein the intensities of the sequence of x-ray emissions are dynamically adjusted based on attenuation of the sequence of fan beams by the target object.

6. The computed tomography x-ray imaging system of claim 2, further comprising an elongated bowtie filter covering the plurality of x-ray sources to generate a predefined intensity profile along the rotation axis in each of the sequence of fan beams.

7. The computed tomography x-ray imaging system of claim 1, further comprising an elongated bowtie filter covering the plurality of x-ray sources to generate a predefined intensity profile along the rotation axis in each of the sequence of fan beams.

8. The computed tomography x-ray imaging system of claim 1, wherein:
the gantry frame together with the x-ray source array, the elongated linear detector array, and the multi-slot collimator is configured to rotate around the rotation axis to detect projections of multiple sequences of fan beams through the target object; and
the circuitry is further configured to reconstruct a computed tomography x-ray image of the target object from the detected projections.

9. The computed tomography x-ray imaging system of claim 1, wherein the plurality of x-ray sources are disposed to form a curved line in a plane perpendicular to the rotation axis, the curved line having a center located at the elongated linear detector array.

10. The computed tomography x-ray imaging system of claim 1, wherein the plurality of x-ray sources comprise a linear array of x-ray sources.

11. The computed tomography x-ray imaging system of claim 1, wherein the multi-slot collimator comprises a plurality of slot openings each associated with one of the plurality of x-ray sources for generating one of the sequence of fan beams.

12. The computed tomography x-ray imaging system of claim 1, wherein the activation of the plurality of x-ray sources repeats with a repetition rate matching a frame rate of the elongated linear detector array.

13. A method for producing a computed tomography x-ray image of a target object, the method comprising:
sequentially controlling an activation of a plurality of x-ray sources to generate a sequence of x-ray emissions while rotating a gantry frame around the target object with respect to a rotation axis, the plurality of x-ray sources being disposed on the gantry frame and aligned in a direction perpendicularly to the rotation axis;
collimating the sequence of x-ray emissions into a sequence of fan beams;
projecting the sequence of fan beam through the target object into an elongated linear detector array, the elongated linear detector array being disposed on the gantry frame opposing the plurality of x-ray sources with respect to the rotation axis and comprising a plurality of x-ray detectors linearly extending along the rotation axis, and the sequence of fan beams intersecting the target object to form a tetrahedron imaging volume; and
measuring, while rotating the gantry frame, amounts of multiple sequences of fan beams after in order to produce the computed tomography x-ray image of the target object.

14. The method of claim 13 further comprising dynamically adjusting intensities of the sequence of x-ray emissions individually.

15. The method of claim 14, wherein the intensities of the sequence of x-ray emissions are dynamically adjusted to accumulatively favor a projected amount of x-rays passing through a region of interest in the target object.

16. The method of claim 15, wherein control signals for adjusting the intensities of the sequence of x-ray emissions is based on a precomputation according to the region of interest in the target object

17. The method of claim 14, further comprising generating a predefined intensity profile along the rotation axis in each of the sequence of fan beams using an elongated bowtie filter disposed to cover the plurality of x-ray sources.

18. The method of claim 13, further comprising generating a predefined intensity profile along the rotation axis in each of the sequence of fan beams using an elongated bowtie filter disposed to cover the plurality of x-ray sources.

19. The method of claim 13, wherein the plurality of x-ray sources are disposed to form a curved line in a plane perpendicular to the rotation axis, the curved line having a center located at the elongated linear detector array.

20. The method of claim 13, wherein the activation of the plurality of x-ray sources repeats with a repetition rate matching a frame rate of the elongated linear detector array.
